(19) United States
(12) Patent Application Publication
(10) Pub. No.: US 2025/0064024 A1
(43) Pub. Date: Feb. 27, 2025

(54) NON-DESTRUCTIVE QUANTITATIVE DETERMINATION OF AT LEAST ONE PERFORMANCE INDICATOR IN REARING A POPULATION OF LIVE INSECTS IN A COMPLEX MEDIUM

(71) Applicant: Ynsect, Évry-Courcouronnes Cedex (FR)

(72) Inventors: Bénédicte LORRETTE, Montlhéry (FR); Jérôme RICHARD, Alfortville (FR); Laura DARAI, Monthléry (FR); Benjamin ARMENJON, Paris (FR); Maroua NOURI, Bezons (FR)

(73) Assignee: Ynsect, Évry-Courcouronnes Cedex (FR)

(21) Appl. No.: 18/724,702

(22) PCT Filed: Dec. 27, 2022
(86) PCT No.: PCT/FR2022/052513

§ 371 Date: Jun. 27, 2024

(30) Foreign Application Priority Data
Dec. 28, 2021 (FR) FR2114582

(51) Int. Cl. A01K 29/00; A01K 67/033; G01N 21/27; G01N 33/483

(52) U.S. Cl. CPC A01K 29/005; A01K 67/033; G01N 21/27; G01N 33/4833

(57) ABSTRACT

The present invention relates to a non-destructive method for quantitatively determining at least one performance indicator in rearing a population of live insects in a complex medium, the method comprising the following steps: (i) a step of irradiating the complex medium comprising the population of live insects with a light comprising one or more wavelengths within a range from 420 nm to 2500 nm, (ii) a step of collecting the light reflected by the complex medium to obtain a reflectance spectrum of the medium; and (iii) a step of correlating said spectrum with a quantitative value of at least one performance indicator. The present invention also relates to the uses of the method and to the use of a spectrophotometer to quantitatively determine at least one performance indicator of a population of live insects in a complex medium.

### PRIOR ART

The present invention belongs to the field of analysis, and more particularly, it relates to a non-destructive method allowing to determine a quantitative value of at least one performance indicator.

There has been a significant rise in the rearing of insects over the last few years. The rearing of insects is of interest for numerous reasons, whether it is for the agro-industry, since insects constitute a source of proteins of interest, or for other industrial fields, since insects are also a source of chitin, transformable into chitosan by deacetylation, the uses of which are numerous for example such as in cosmetics, in the medical and pharmaceutical field, in nutrition and in the food industry, or for the treatment of water.

It is therefore necessary to put in place industrial models adapted to insects, allowing to be able to monitor their rearing in an automated manner, in order to maintain optimal growth conditions.

This monitoring is possible via the quantification of performance indicators (Key Performance Indicators or KPIs).

“Performance indicator” (Key Performance Indicator or KPI) means, preferably, a piece of information intended to be expressed as a figure which allows to measure and evaluate the results of one or more actions, to monitor the evolution of a performance and/or to analyse a situation.

The methods currently used are manual and fastidious (sorting and counting of the populations of insects, visual inspection of the rearing tanks) and incompatible with a rearing on an industrial scale. Moreover, these methods lack precision and can sometimes damage the insects during growth, in particular during the manipulation of the latter.

There is therefore a need to automate the monitoring of growth of a population of live insects and the monitoring of the equipment for sorting and feeding a population of live insects.

The present invention overcomes these technical problems and relates to a non-destructive method for determining a quantitative value of at least one performance indicator. Moreover, the present inventors have shown in a surprising and unexpected manner that the method according to the invention allows to determine a quantitative value of at least one performance indicator with very low uncertainty in a complex medium.

### SUMMARY OF THE INVENTION

The present invention relates to a non-destructive method for quantitatively determining at least one performance indicator of a rearing of a population of live insects in a complex medium, said method comprising the following steps:
(i) a step of irradiating the complex medium including the population of live insects with a light including one or more wavelengths comprised in a range going from 420 nm to 2500 nm,
(ii) a step of collecting the light reflected by the complex medium in order to obtain a reflectance spectrum of the medium, and
(iii) a step of correlating said spectrum to a quantitative value of at least one performance indicator.

The present invention also relates to the use of the method as defined above to carry out the monitoring of growth of the population of live insects, to determine the efficiency of a sorting machine and/or to determine the efficiency of the equipment for feeding the population of live insects.

The present invention also relates to the use of a spectrophotometer for quantitatively determining at least one performance indicator of a rearing of a population of live insects in a complex medium.

### DETAILED DESCRIPTION

In the context of the present application, unless otherwise indicated, the ranges of values indicated are understood to include the endpoints.

A first object relates to a non-destructive method for quantitatively determining at least one performance indicator of a rearing of a population of live insects in a complex medium, said method comprising the following steps:
(i) a step of irradiating the complex medium including the population of live insects with a light including one or more wavelengths comprised in a range going from 420 nm to 2500 nm,
(ii) a step of collecting the light reflected by the complex medium in order to obtain a reflectance spectrum of the medium, and
(iii) a step of correlating said spectrum to a quantitative value of at least one performance indicator.

“Non-destructive method” means, preferably, a set of non-invasive steps allowing to preserve the integrity of the population of live insects.

“Insect” means, preferably, an insect in any stage of development, such as the adult, larval, nymph and/or egg stage, preferably adult, larval and/or nymph stage. This term thus covers any stage of evolution (also called growth or development stage) from the egg or ootheca to the adult insect, including the larva and the nymph such as the pupa.

“Population of insects” means, preferably, a set of at least two insects of an identical or different species and/or at an identical or different stage of development.

In particular, a population of insects according to the present invention relates to a population of individualised insects. Therefore, the population of insects as intended by the invention does not correspond to an insect that is incubating eggs inside its abdomen (ovoviviparous insect).

Preferably, a population of insects, and more particularly the population of live insects, is a set of at least two insects of an identical species at an identical stage of development. Examples of population of insects include, but are not limited to, a set of larvae, a set of nymphs or a set of adult insects.

“Larva” means, preferably, the larval stage of the insects and thus the juvenile stage of the insect, which includes the maggot for dipterans and the caterpillar for lepidopterans, as well as the apteral stages for orthopterans. This term covers any stage of development of the larva.

“Nymph” means, preferably, the metamorphosis stage of the insect and in particular the intermediate stages between the larva and the imago, which includes the pupa for the dipterans, the nymph for the coleopterans, the chrysalis for the lepidopterans and, if necessary, an intermediate stage during which certain physiological (prepupa) or behavioural modifications of the individuals appear, such as a significant sclerification of the cuticle for the dipterans.

“Adult insect” (or imago) means, preferably, the mature stage of the insect, and in particular the last stage of development of the insect, which ensures the reproduction of the species.

Advantageously, the insects are chosen from the group of the beetles, the dipterans, the lepidopterans, the orthopterans, the hymenopterans, the neuropterans, the dictyopterans encompassing in particular the blattopterans, including isopterans, and the mantopterans, the phasmatopterans, the hemipterans, the heteropterans, the ephemeropterans, the mecopterans, and the mixtures thereof, preferably, from the group of the beetles, the dipterans, the lepidopterans, the orthopterans and the mixtures thereof, more preferably the insect belongs to the group of the beetles. Preferably, the dipterans belong to the suborder of the Brachycera. Preferably, the lepidopterans belong to the suborder of the Ditrysia, more preferably to the superfamily of the Pyraloidea. Preferably, the neuropterans belong to the suborder of the Hemerobiiformia. More preferably, the neuropterans belong to the families of the Mantispidae, Ithonidae, Chrysopidae, Hemerobiidae, or the mixtures thereof. Preferably, the beetles belong to the suborder of the Cucujiformia, in particular to the families of the Tenebrionidae, Coccinellidae, Cerambycidae, Dryophthoridae, or mixtures thereof. More preferably, the beetles are chosen from Tenebrio molitor, Alphitobius diaperinus, Zophobas morio, Tenebrio obscurus, Tribolium castaneum, Rhynchophorus ferrugineus, and the mixtures thereof, even more preferably Tenebrio molitor, Alphitobius diaperinus, and the mixtures thereof. The insects at which the invention is aimed thus preferably belong to the group of the beetles and more particularly to the family of the Tenebrionidae. Preferably, the insects at which the invention is aimed belong to the species Tenebrio molitor and/or Alphitobius diaperinus.

“Complex medium” means, preferably:
an assembly comprising at least two, preferably at least three, different components other than the population of live insects, and/or
at least one insect at at least one different stage of development than at least one insect of the population of live insects, and/or
at least one dead insect.

“Assembly comprising at least two, preferably at least three, different components” means, preferably, an assembly comprising at least two, preferably at least three, components composed at least partly of identical or similar molecules but at different concentrations.

“Identical or similar molecules” means, preferably, chemical molecules having identical or similar chemical structures.

For example, these are molecules having in common chemical structures having the same vibrations, during the excitation of the latter by the infrared source, of the hydrogen atom as those of the molecule family measured (proteins, lipids, saccharides, chitins, water). For example, if the hydrogen is bonded to a nitrogen (via an N—H2-COOH group), then the molecules belong to the family of the proteins.

Thus, the proteins have identical or similar chemical structures.

Likewise, the monosaccharides, oligo and polysaccharides have identical or similar chemical structures; the triglycerides and fatty acids have identical or similar chemical structures; chitin and the chitin derivatives have identical or similar chemical structures.

“Chitin derivatives” means, preferably, derivatives of polysaccharides including N-acetyl-glucosamine units and D-glucosamine units, in particular the chitin-polypeptide copolymers (sometimes designated by the name “chitin-polypeptide composite”).

Indeed, in the complex medium according to the invention, each component can in particular include two or more of the compounds chosen from:
the proteins;
the monosaccharides, oligo and polysaccharides;
the triglycerides and fatty acids; and/or
chitin and the chitin derivatives.

More particularly, each compound above is present in at least two chosen from the components of the complex medium and the population of live insects.

Preferably, the complex medium is a medium for rearing insects.

Examples of components can be, but are not limited to, a substrate, and/or dead insects of an identical or different species than that of the population of live insects, and/or live insects of a different species than at least one insect of the population of live insects, preferably than that of the population of live insects and/or live insects at a different stage of development than at least one insect of the population of live insects, and/or droppings (also called frass), and/or contaminants.

“Substrate” means, preferably, a nutritive substrate.

“Nutritive substrate” means, preferably, a nutritive substrate or a mixture of nutritive substrates, intended to be consumed by the insects. This can be a solid or liquid nutritive substrate. The solid nutritive substrate can be a mixture of solid products or co-products coming from the processing of cereals, of oleaginous plants, of oleaproteaginous plants and/or of proteaginous plants. This substrate can be supplemented with vitamins or with minerals. “Coproduct” preferably means an inevitable material created during a process of manufacturing a product of interest. The solid substrate can be a product or coproduct coming from the processing of wheat (common wheat, durum wheat), corn, barley, rice, triticale, oats, sorghum, rye, spelt, millet, quinoa, buckwheat, rape, sunflower, flax, soybeans and/or peas. Preferably, the solid substrate is a product or coproduct coming from the processing of wheat, more preferably, the substrate is wheat bran and/or wheat middlings. Alternatively, dried distilled grains with solubles can be used.

“Droppings” means, preferably, droppings (faeces) of insects regardless of their stage of development.

“Contaminants” means, preferably, an undesirable living organism such as a bacterium or a fungus, or another insect such as a moth, and/or an undesirable element produced by the latter such as moth silk. Preferably, the contaminant is a moth and/or a moth silk. More preferably, the contaminant is a moth silk.

Advantageously, the complex medium comprises at least two, preferably at least three, components different from the population of live insects.

Advantageously, the at least two, preferably at least three, different components are chosen from a substrate and droppings.

Advantageously, the complex medium comprises at least one insect at at least one different stage of development than at least one insect of the population of live insects.

Advantageously, the complex medium comprises at least one insect at at least one different stage of development than the population of live insects, said population of live insects thus being a population of insects at a given stage of development, more particularly, said population of live insects being a population of insects of a given species at a given stage of development. Such a case allows in particular to distinguish a population of larvae from a population of nymphs or a population of nymphs from a population of adults.

Advantageously, the complex medium comprises at least one dead insect.

“Quantitative determination” means, preferably, the determination of a quantitative value.

Preferably, “performance indicator” or “KPI” (“Key Performance Indicator”) of a rearing of a population of live insects means an indicator relative to the population of live insects or to a component of the complex medium or to a characteristic of the population of live insects or to a characteristic of a component of the complex medium at a given time.

“Quantitative value of a performance indicator” means, preferably, a quantitative value associated with the population of live insects (for example, a weight percentage) or with a component of the complex medium (for example, a weight percentage) or with a characteristic of the population of live insects (for example, an individual mean mass) or with a characteristic of a component of the complex medium (for example, a weight percentage of moth silk aggregate) at a given time. The quantitative value of a component of the complex medium can for example be the quantity of said component comprised in the complex medium, and preferably the mass quantity such as a weight percentage of said component relative to the total weight represented by the complex medium and the population of live insects.

Examples of a quantitative value of a performance indicator comprise, but are not limited to, the quantitative value of a given population of insects in the complex medium expressed in mass or in weight percentage relative to the total weight represented by the complex medium and the population of live insects, the quantitative value of a substrate in the complex medium expressed in mass or in weight percentage relative to the total weight represented by the complex medium and the population of live insects, the quantitative value of droppings in the complex medium expressed in mass or in weight percentage relative to the total weight represented by the complex medium and the population of live insects, the quantitative value of contaminants in the complex medium expressed in mass or in weight percentage relative to the total weight represented by the complex medium and the population of live insects, the individual mean mass (IMM) of the insects in the complex medium expressed in mass.

Thus, in a surprising and unexpected manner, the present inventors have developed a method allowing to quantify in a complex medium specific populations of insects and other components of the medium. For example, the method according to the invention allows to quantify in a complex medium insects at various stages of development such as larvae, nymphs and/or adult insects, and dead insects.

“Individual mean mass” (or “IMM”) means, preferably, the individual mean mass of the insects.

Advantageously, the performance indicator is chosen from a quantification of a population of insects, of a substrate, of the droppings, of a contaminant and/or an individual mean mass of the insects, preferably a quantification of a population of insects, an individual mean mass of the insects and/or of the droppings, and even more preferably a quantification of an insect population such as dead insects and/or droppings.

Advantageously, the performance indicator is a quantification of the droppings.

Advantageously, the performance indicator is a quantification of a population of insects.

Advantageously, the population of insects consists of the dead insects.

Advantageously, the population of insects consists of insects at a given stage of development, the insects being advantageously living.

“Irradiation step” means, preferably, the emission of a light including one or more wavelengths onto the medium.

It s noted that according to the invention, the irradiation step is carried out on the complex medium including the population of live insects, that is to say on the complex medium in the presence of the population of live insects.

Advantageously, the irradiation step (i) is carried out with a light including one or more wavelengths comprised in the range going from 800 nm to 2200 nm, preferably from 1000 to 1950 nm, and even more preferably from 1100 to 1650 nm.

“Collection step” means, preferably, the acquisition via a detector of the energy intensity reflected by the medium according to the wavelengths of the light emitted in step (i). The light of step (i) is advantageously a decomposed light, preferably, a light decomposed into monochromatic lights.

“Monochromatic light” means, preferably, a light comprising one or more wavelengths of the same colour.

Advantageously, a monochromatic light comprises a set of wavelengths, the shortest wavelength and the longest wavelength of which are spaced apart by at most 50 nm, preferably, by at most 25 nm, more preferably by at most 15 nm, even more preferably, by at most 10 nm, such as approximately 8.75 nm.

Advantageously, the complex medium is irradiated with one or more monochromatic lights comprised in a range going from 420 nm to 2500 nm, preferably from 800 to 2200 nm, preferably from 1000 to 1950 nm, and even more preferably from 1100 to 1650 nm.

In the case of several monochromatic lights, steps (i) and (ii) are carried out several times with each monochromatic light emitted successively.

According to a preferred embodiment, several irradiation steps (i) are carried out successively so as to irradiate the sample with a range of light, the wavelengths of which are comprised between 420 nm and 2500 nm, preferably from 800 to 2200 nm, preferably from 1000 to 1950 nm, and even more preferably from 1100 to 1650 nm, a collection step (ii) being carried out for each irradiation step.

“Range of light” means, preferably, a sum of lights covering all of the wavelengths comprised between the two endpoints of the range.

For example, in order to obtain a range of light going from X to Y nm, several sequential irradiation steps (i) can be carried out via the emission of sequential monochromatic lights, the first monochromatic light starting at Xnm, and the following monochromatic lights being emitted with a defined step, for example 0.5 nm (in this case, the second monochromatic light is emitted at X+0.5 nm, the third at X+1 nm, etc.), up to Y. Preferably, this step is between 0.2 nm and 10 nm, more preferably, between 0.5 nm and 5 nm, even more preferably, approximately 0.5 nm.

“Reflectance spectrum” means, preferably, a spectrum representing the energy intensity reflected by the medium according to the wavelengths with which said medium was irradiated.

Advantageously, steps (i) and (ii) of the method according to the invention are repeated several times, and preferably at least once, in order to improve the quality of the reflectance spectrum of the medium.

Preferably, after the collection step (ii) of the method according to the invention, a diffuse reflectance spectrum of the medium is obtained.

“Diffuse reflectance spectrum” preferably means a reflectance spectrum representing the energy intensity reflected by the medium having the particularity of reflecting and dispersing the energy intensity in all the directions of space, according to the wavelengths with which said medium was irradiated.

Advantageously, steps (i) and (ii) of the method according to the invention are carried out using a spectrophotometer, in particular using a near-infrared spectrophotometer (also called NIRS spectrometer for “near-infrared spectroscopy”), for example such as the NIRS XDS Rapid Content Analyzer from Metrohm, the NIRS Analyzer Processor from Metrohm or the NIRS XDS Process Analyzer from Metrohm.

The operation of a spectrophotometer is known to a person skilled in the art: a light, in general polychromatic, coming for example from a tungsten or halogen lamp, is sent through an input slit in order to generate a spatially coherent light. This light then arrives onto a diffraction grating, thus leading to the spatial separation of the wavelengths forming it. Via diffuse reflection, a part of the signal is then sent back to the detectors. An output slit allows to limit the passage of the wavelengths arriving at the sample for the irradiation step; this output slit thus defines a bandwidth of a given size. Consequently, several wavelengths can arrive simultaneously at the sample, according to the size of the bandwidth. The wider the output slit, the larger the number of wavelengths arriving at the sample, and thus the more the resolution of the reflectance spectrum is altered. However, the smaller the width of the output slit, the finer the resolution of the reflectance spectrum, but the more the signal-to-noise ratio is degraded. In order to allow the sequential passage of the spatially separated wavelengths in the output slit, the diffraction grating can be mounted on an encoding motor which allows to vary its angular position, and thus to define an acquisition step, or the apparatus is equipped with a diode array.

Preferably, the acquisition step of the spectrophotometer is between 0.2 nm and 8 nm, more preferably, between 0.5 nm and 4 nm, even more preferably, approximately 0.5 nm.

Preferably, the bandwidth of the spectrophotometer is between 1 nm and 50 nm, more preferably, between 5 and 20 nm, even more preferably, between 8 nm and 10 nm, such as for example approximately 8.75 nm.

The spectrophotometer can comprise one or more detectors, preferably at least two detectors (also called sensors).

Preferably, the spectrophotometer comprises a detector made of silicon in order to detect the energy intensity reflected by the sample for wavelengths between 400 nm and 1100 nm, and a detector made of lead sulphate in order to detect the energy intensity reflected by the sample for wavelengths between 1100 nm and 2500 nm. Usually, in the case in which it is desired to obtain a reflectance of diffuse spectrum the sample, the spectrophotometer comprises a hemispherical reflector dome placed above the sample in order to direct all the reflected light towards the detector.

According to a preferred embodiment, the steps (i) of irradiation and (ii) of collection are carried out using a spectrophotometer and repeated several times, advantageously while modifying the position of the sample. The succession of steps (i) and (ii) is routinely called a scan.

Preferably, steps (i) and (ii) are repeated at least 12 times, more preferably at least 16 times, even more preferably at least 24 times for example such as 32 times (or 32 scans). Likewise, the position of the sample is preferably modified at least 2 times, more preferably at least 4 times, for example such as 8 times. Even more preferably, steps (i) and (ii) are repeated 32 times per position of the sample and the position of the sample is modified between 4 and 8 times.

Steps (i) and (ii) of the method according to the invention can be carried out when the complex medium is immobile or moving.

When the complex medium is moving, this is called dynamic mode. On the contrary, when the complex medium is immobile, this is called static mode.

Whether steps (i) and (ii) are carried out according to a static mode or according to a dynamic mode, the spectrophotometer can be integrated into an optical device including a motorised mobile support to which the spectrophotometer is linked. This allows to limit the number of spectrophotometers necessary to acquire data over a free surface of the complex medium sufficient to be representative of the entirety of the free surface of the complex medium. The motorised mobile support, in association if necessary with a conveying device on which the rearing tank containing the complex medium is placed, allows to create a relative movement between the spectrophotometer and said complex medium. Thus, the field of acquisition of the spectrophotometer sweep during steps (i) and (ii) a part of the total free surface of the complex medium much greater than the surface over which data could be acquired with a fixed sensor.

In dynamic mode, and as a simple example, the mobile support can include a rotary arm parallel to the upper surface of the conveying device (for example a belt conveyor) on which the tank containing the complex medium is placed. The arm is thus also substantially parallel to the free surface of the complex medium. The optical device is configured so that the rotary arm carries out one or more rotations (for example three rotations) while a rearing tank passes under the sensor at a constant speed imposed by the conveying device on which it is placed.

In static mode, and as a simple example, the mobile support includes means for translations of the spectrophotometer in two orthogonal directions, called longitudinal and transverse, in a plane parallel to the free surface of the complex medium. The tank containing the complex medium is positioned and immobilised under the spectrophotometer. The optical device is configured so that the zone scanned by the acquisition field of the spectrophotometer has a “racetrack” shape, at the surface of the complex medium. A racetrack shape is an oblong shape including two parallel longitudinal straight trajectories connected by turns and/or transverse portions. To maximise the surface scanned by the acquisition field of the spectrophotometer, the scanned zone can have the shape of two racetracks, namely a scanned racetrack-shaped zone included in another scanned racetrack-shaped zone having larger dimensions.

“Correlation step” means a step allowing to determine a quantitative value of a given performance indicator according to the reflectance spectrum of the medium. The quantitative value of a given performance indicator can thus be correlated to the data of said spectrum via the reflectance of the medium measured at one or more specific wavelengths.

Thus, the step (iii) of correlation is carried out while using the totality, or a part, or parts of the reflectance spectrum. Preferably, the step (iii) of correlation is carried out while using the totality of the reflectance spectrum.

Advantageously, the step (iii) of correlation is carried using a predictive model previously established for this component.

Preferably, said model was previously established by a regression analysis.

A regression analysis is a statistical method that allows to model relationships between various variables, more particularly between a dependent variable and one or more independent variables, the value of the independent variables affecting the value of the dependent variable. At the end of the analysis, a predictive model is thus established and is shown by an equation. In the context of the present invention, a dependent variable is a quantitative value of a performance indicator and an independent variable is the quantity of light reflected by the insect of the population of live insects or a component of the complex medium for a wavelength or over a given range of wavelengths.

Preferably, the regression analysis is a simple linear regression, a multilinear regression, or a partial least squares regression (called PLS). More preferably, the regression analysis is a PLS regression. These regression analyses are well known to a person skilled in the art and can for example be carried out using a commercial piece of software, preferably using the software Vision marketed by Metrohm.

In order to carry out a regression analysis, several samples of medium must be constituted, preferably, at least 50 samples, more preferably, at least 100 samples, even more preferably, at least 150 samples for example such as 180. Then, each sample is irradiated with a light including one or more wavelengths comprised in one or more ranges going from 420 nm to 2500 nm, preferably from 800 to 2200 nm, preferably from 1000 to 1950 nm and even more preferably going from 1100 nm to 1650 nm, then the light reflected by each sample is collected in order to obtain a reflectance spectrum for each sample. Each sample is also analysed by a reference method in order to determine a reference quantitative value in a population of live insects or in a given component of the complex medium. Preferably, this reference method involves manually separating the various components of the complex medium and the population of live insects then weighing them. Alternatively and even more preferably, for artificial complex mediums, the mass of each component present in the medium is known, it is not therefore necessary to manually separate the various components of the medium. The same applies to the population of live insects. Method examples are described in the examples below.

The PLS regression can be applied to several wavelengths, or even onto a large spectral range or even onto the entire reflectance spectrum.

Moreover, the PLS regression to allows determine independent variables that cannot be observed (for example, which have little to no meaning physically), they are called latent variables or factors. The PLS regression thus allows the creation of new variables.

According to a preferred embodiment, the regression analysis is a PLS regression that involves the choice of a number of factors to be included in the equation of the predictive model. This choice of the number of factors is important since, if there are not enough factors, this leads to an under-modelling and to a degradation of the accuracy of the model, and inversely, too many factors lead to an over-modelling and to a degradation of the robustness of the model. Advantageously, the optimal number of factors to be used for the PLS regression is determined by calculating the predicted residual error sum of squares (PRESS) for each number of factors, the PRESS value thus representing the prediction error and needing to be minimal. Preferably, the PRESS value of each number of factors is calculated via an external validation set or a cross-validation, more preferably, via a cross-validation.

Thus, the number of factors having a minimal PRESS value is chosen to establish the equation of the predictive model. Since the PRESS value depends on the component of the medium, the minimal value to be chosen is not the same according to the model to be established for a given component of the medium.

Preferably, during the cross-validation, all the data of the samples having participated in the establishment of a predictive model is grouped together, then the data for each sample is removed in turns and a model is created with the spectra and values, determined during the associated reference analyses, of the remaining samples. The cross-validation is described in more detail in the examples below.

Advantageously, the method further comprises, after the step (ii) of collection and before the step (iii) of correlation, a step of mathematical processing of the reflectance spectrum comprising a first derivative, a second derivative, a processing of the Savinsky type and/or a normalisation of the SNV type (“Standard Normal Variate”).

Preferably, the step of mathematical processing is a normalisation of the SNV type followed by a first or second derivative or a processing of the Savinsky type followed by a first or second derivative.

Advantageously, the mass percentage of insects in the larval stage in a complex medium of the method according to the invention is between 0% and 90%, preferably between 20% and 85%, and more preferably between 25% and 80% of the total weight of the sample of the complex medium and of the live insects.

Advantageously, the mass percentage of insects in the adult stage in a complex medium of the method according to the invention is between 0% and 100%, preferably between 10% and 90%, and more preferably between 20% and 80% of the total weight of the sample of the complex medium and of the live insects.

Advantageously, the mass percentage of insects in the nymph stage in a complex medium of the method according to the invention is between 0% and 50%, preferably between 0.1% and 20% of the total weight of the sample of the complex medium and of the live insects.

Advantageously, the mass percentage of dead insects in a complex medium of the method according to the invention is between 0% and 50%, preferably between 0.1% and 40% of the total weight of the sample of the complex medium and of the live insects.

Advantageously, the mass percentage of substrate in a complex medium of the method according to the invention is between 0% and 60%, preferably between 5% and 50% of the total weight of the sample of the complex medium and of the live insects.

Advantageously, the mass percentage of droppings in a complex medium of the method according to the invention is between 0% and 50%, preferably between 5% and 40% of the total weight of the sample of the complex medium and of the live insects.

Advantageously, the mass percentage of contaminants such as moth silk aggregates in a complex medium of the method according to the invention is between 0% and 90%, preferably between 0% and 50%, and more preferably between 0% and 40% of the total weight of the sample of the complex medium and of the live insects.

Advantageously, the individual mean mass of the insects in a complex medium of the method according to the invention is calibrated according to two subgroups:
a group in which the mass is estimated between 0.5 mg and 50 mg, preferably between 0.7 mg and 48 mg, more preferably between 1 mg and 45 mg,
another subgroup between 35 and 200 mg, preferably between 38 and 185 mg, more preferably between 40 mg and 175 mg.

The present invention also relates to the use of the method according to the invention to carry out the monitoring of growth (or of evolution or of development) of the population of live insects and preferably the monitoring of growth (or of evolution or of development) of the population of live insects for the control of quality of rearing of insects, to determine the efficiency of a machine for sorting, and preferably for sorting insects, and/or to determine the efficiency of the equipment for feeding the population of live insects, preferably using a spectrophotometer as defined above.

The present invention also relates to the use of a spectrophotometer, for the quantitative determination of at least one performance indicator in a complex medium.

Advantageously, the spectrophotometer is as defined above.

Advantageously, the performance indicator is as defined above.

Advantageously, the spectrophotometer is used to carry out the monitoring of growth (or of evolution or of development) of the population of live insects, and preferably the monitoring of growth (or of evolution or of development) of the population of live insects for the control of quality of rearing of the population of live insects, to determine the efficiency of a machine for sorting, and preferably for sorting the population of live insects, and/or to determine the efficiency of the equipment for feeding the population of live insects.

The present invention is illustrated, in a non-limiting manner, by the examples as well as the drawings.

### DESCRIPTION OF THE DRAWINGS

FIG. 1 includes two diagrams: the one on the left (A) shows the mass quantity of live insects in the larval stage in the sampling A calculated from the learning set according to the mass quantity of live insects in the larval stage determined by the reference analyses, and the one on the right (B) shows the mass quantity of live insects in the larval stage in the sampling A calculated from the external validation set according to the quantity of live insects in the larval stage determined by the reference analyses, the mass quantities being expressed as a percentage by weight of the total weight of the sample.

FIG. 2 includes two diagrams: the one on the left (A) shows the mass quantity of live insects in the larval stage in the sampling B calculated from the learning set according to the mass quantity of live insects in the larval stage determined by the reference analyses, and the one on the right (B) shows the mass quantity of live insects in the larval stage in the sampling B calculated from the external validation set according to the quantity of live insects in the larval stage determined by the reference analyses, the mass quantities being expressed as a percentage by weight of the total weight of the sample.

FIG. 3 includes two diagrams: the one on the left (A) shows the mass quantity of live insects in the nymph stage in the sampling B calculated from the learning set according to the mass quantity of live insects in the nymph stage determined by the reference analyses, and the one on the right (B) shows the mass quantity of live insects in the nymph stage in the sampling B calculated from the external validation set according to the quantity of live insects in the nymph stage determined by the reference analyses, the mass quantities being expressed as a percentage by weight of the total weight of the sample.

FIG. 4 includes two diagrams: the one on the left (A) shows the mass quantity of live insects in the adult stage in the sampling C calculated from the learning set according to the mass quantity of live insects in the adult stage determined by the reference analyses, and the one on the right (B) shows the mass quantity of live insects in the adult stage in the sampling C calculated from the external validation set according to the quantity of live insects in the adult stage determined by the reference analyses, the mass quantities being expressed as a percentage by weight of the total weight of the sample.

FIG. 5 includes two diagrams: the one on the left (A) shows the mass quantity of live insects in the nymph stage in the sampling C calculated from the learning set according to the mass quantity of live insects in the nymph stage determined by the reference analyses, and the one on the right (B) shows the mass quantity of live insects in the nymph stage in the sampling C calculated from the external validation set according to the quantity of live insects in the nymph stage determined by the reference analyses, the mass quantities being expressed as a percentage by weight of the total weight of the sample.

FIG. 6 includes two diagrams: the one on the left (A) shows the mass quantity of live insects in the adult stage in the sampling D calculated from the learning set according to the mass quantity of live insects in the adult stage determined by the reference analyses, and the one on the right (B) shows the mass quantity of live insects in the adult stage in the sampling D calculated from the external validation set according to the mass quantity of live insects in the adult stage determined by the reference analyses, the mass quantities being expressed as a percentage by weight of the total weight of the sample.

FIG. 7 includes two diagrams: the one on the left (A) shows the mass quantity of droppings in the sampling A calculated from the learning set according to the mass quantity of droppings determined by the reference analyses, and the one on the right (B) shows the mass quantity of droppings in the sampling A calculated from the external validation set according to the mass quantity of droppings determined by the reference analyses, the mass quantities being expressed as a percentage by weight of the total weight of the sample.

FIG. 8 includes two diagrams: the one on the left (A) shows the mass quantity of droppings in the sampling B calculated from the learning set according to the mass quantity of droppings determined by the reference analyses, and the one on the right (B) shows the mass quantity of droppings in the sampling B calculated from the external validation set according to the mass quantity of droppings determined by the reference analyses, the mass quantities being expressed as a percentage by weight of the total weight of the sample.

FIG. 9 includes two diagrams: the one on the left (A) shows the mass quantity of a nutritive substrate in the sampling A calculated from the learning set according to the mass quantity of a nutritive substrate determined by the reference analyses, and the one on the right (B) shows the mass quantity of a nutritive substrate in the sampling A calculated from the external validation set according to the mass quantity of a nutritive substrate determined by the reference analyses, the mass quantities being expressed as a percentage by weight of the total weight of the sample.

FIG. 10 includes two diagrams: the one on the left (A) shows the mass quantity of a nutritive substrate in the sampling B calculated from the learning set according to the mass quantity of a nutritive substrate determined by the reference analyses, and the one on the right (B) shows the mass quantity of a nutritive substrate in the sampling B calculated from the external validation set according to the mass quantity of a nutritive substrate determined by the reference analyses, the mass quantities being expressed as a percentage by weight of the total weight of the sample.

FIG. 11 includes two diagrams: the one on the left (A) shows the mass quantity of a nutritive substrate in the sampling C calculated from the learning set according to the mass quantity of a nutritive substrate determined by the reference analyses, and the one on the right (B) shows the mass quantity of a nutritive substrate in the sampling C calculated from the external validation set according to the mass quantity of a nutritive substrate determined by the reference analyses, the mass quantities being expressed as a percentage by weight of the total weight of the sample.

FIG. 12 includes two diagrams: the one on the left (A) shows the mass quantity of dead insects in the sampling A calculated from the learning set according to the mass quantity of dead insects determined by the reference analyses, and the one on the right (B) shows the mass quantity of dead insects in the sampling A calculated from the external validation according to the mass quantity of dead insects determined by the reference analyses, the mass quantities being expressed as a percentage by weight of the total weight of the sample.

FIG. 13 includes two diagrams: the one on the left (A) shows the mass quantity of dead insects in the sampling B calculated from the learning set according to the mass quantity of dead insects determined by the reference analyses, and the one on the right (B) shows the mass quantity of dead insects in the sampling B calculated from the external validation set according to the mass quantity of dead insects determined by the reference analyses, the mass quantities being expressed as a percentage by weight of the total weight of the sample.

FIG. 14 includes two diagrams: the one on the left (A) shows the mass quantity of dead insects in the sampling C calculated from the learning set according to the mass quantity of dead insects determined by the reference analyses, and the one on the right (B) shows the mass quantity of dead insects in the sampling C calculated from the external validation set according to the mass quantity of dead insects determined by the reference analyses, the mass quantities being expressed as a percentage by weight of the total weight of the sample.

FIG. 15 includes two diagrams: the one on the left (A) shows the mass quantity of dead insects in the sampling D calculated from the learning set according to the mass quantity of dead insects determined by the reference analyses, and the one on the right (B) shows the mass quantity of dead insects in the sampling D calculated from the external validation set according to the mass quantity of dead insects determined by the reference analyses, the mass quantities being expressed as a percentage by weight of the total weight of the sample.

FIG. 16 includes two diagrams: the one on the left (A) shows the mass quantity of contaminants (moth silk aggregates) in the sampling E calculated from the learning set according to the mass quantity of contaminants (moth silk aggregates) determined by the reference analyses, and the one on the right (B) shows the mass quantity of contaminants (moth silk aggregates) in the sampling E calculated from the external validation set according to the mass quantity of contaminants (moth silk aggregates) determined by the reference analyses, the mass quantities being expressed as a percentage by weight of the total weight of the sample.

FIG. 17 includes two diagrams: the one on the left (A) shows the individual mean mass (IMM) of the insects calculated from the learning set in the sampling E according to the individual mean mass determined by the reference analyses in the range going from 1.03 mg to 42.86 mg, and the one on the right (B) shows the individual mean mass (IMM) of the insects in the sampling E calculated from the external validation set according to the individual mean mass determined by the reference analyses in the range going from 1.03 mg to 42.86 mg.

FIG. 18 includes two diagrams: the one on the left (A) shows the individual mean mass (IMM) of the insects calculated from the learning set in the sampling E according to the individual mean mass determined by the reference analyses in the range going from 40.85 mg to 173.46 mg, and the one on the right (B) shows the individual mean mass (IMM) of the insects in the sampling E calculated from the external validation set according to the individual mean mass determined by the reference analyses in the range going from 40.85 mg to 173.46 mg.

### EXAMPLES

### Example 1: Creation of the Model Implemented in the Method According to the Invention

### I-Sampling, Acquisition of the Spectra and Reference Analyses

### Samplings

### “Industrial Tank” Samplings

Four samplings (A, B, C and D) were prepared from samples of Tenebrio molitor at various stages of evolution (larva, adult and/or nymph). 2.8 m² rearing tanks were filled artificially by mixing live insects with substrate, dead insects and/or droppings.

The substrate is a nutritive substrate called “INSECTUS” marketed by the company MIJTEN.

The characteristics of each sampling are presented in table 1.

TABLE 1

| | Sampling A | Sampling B | Sampling C | Sampling D |
|---|---|---|---|---|
| Type of | Larvae | Larvae and | Adult | Adult |
| sample | | nymphs | insects | insects* |
| | | | and nymphs | |
| Number of | 180 | 172 | 180 | 180 |
| samples | | | | |
| Range of | 40.3% to | 32.7% to | 31.4% to | 66.7% to |
| mass | 78.6% | 73.2% | 51.3% | 100% |
| percentage | | (larvae) | (adult | |
| of insects | | 4% to | insects) | |
| | | 12.5% | 0% to 5.6% | |
| | | (nymphs ) | (nymphs) | |
| Range of | 8.4% to | 9.4% to | 25% to | |
| mass | 29.9% | 32% | 43.4% | |
| percentage | | | | |
| of | | | | |
| substrate | | | | |
| Range of | 8.4% to | 9.4% to | | |
| mass | 29.9% | 32% | | |
| percentage | | | | |
| of | | | | |
| droppings | | | | |
| Range of | 0% to 4.5% | 0 to 1.7% | 10.6% to | 0% to |
| mass | | | 34.5% | 33.5% |
| percentage | | | (Dead | |
| of dead | | | insects) | |
| insects | | | | |

*This sample was carried out on specific tanks, the bottom of which is provided with a metal grating.

The mass percentages are given relative to the total weight of the components introduced into the tank.

The term “grating” designates a metal grating, the mesh of which allows the laid eggs to pass through while retaining the adults on its surface.

### “Trial Tanks” Sampling

The sampling E was also prepared using samples of Tenebrio molitor in the larval stage. In tanks having a dimension of 0.24 m², Tenebrio molitor insects in the larva stage were reared for 4 weeks in a complex medium comprising nutritive substrate called “INSECTUS” marketed by the company MIJTEN.

During these 4 weeks of rearing, the insects consumed nutritive substrate and produced droppings. Moreover, contaminants such as moths were allowed to infest the insect rearing tanks.

The characteristics of the sampling E are presented in table 2.

TABLE 2

| | Sampling E |
|---|---|
| Type of sample | Larvae |
| Range of mass percentage | 0.5% to 87.8% |
| of moth silk aggregates | |
| Ranges of the IMM | 1.03 mg to 42.86 mg or |
| | 40.85 mg to 173.46 mg |

“Moth silk aggregates” means moth silks agglomerated with a part of the complex medium and/or of the moths.

The sampling E was used in order to quantify two performance indicators, namely the individual mean mass (IMM), expressed in mg, of the insects and the mass percentage of contaminants and in particular the mass percentage of moth silk aggregates. Moreover, 234 samples of this type of sampling E were used in order to develop a model for the “contaminants” KPI while 386 samples of this type of sampling E were used in order to develop a model for the “IMM” KPI over the range 1.03 mg to 42.86 mg and 245 samples over the range 40.85 mg to 173.46 mg.

### Acquisition of the Spectra

### Acquisition of the Spectra on the “Industrial Tank” Samplings

A spectrum in diffuse reflection mode was acquired using an NIRS (for near-infrared spectroscopy) spectrometer, the acquisition conditions being summarised in Table 3.

TABLE 3

| Spectrometer | NIRS Analyzer Pro (Metrohm) |
|---|---|
| Bandwidth | 9.50 ± 0.10 nm |
| Acquisition step | 0.5 nm |
| Integration time | 40 ms |
| Software | Vision 4.1.1.63 (Metrohm) |
| Number of scans | 32 |
| Size of the spot (diameter of the | 70 mm |
| beam) | |
| Acquisition range | 1100-1650 nm |
| Height of the NIRS between the | 20 cm |
| bottom of the tank and the | |
| bottom of the | |
| NIR block | |

For each tank, 4 different positions are analysed via the NIRS Analyzer Pro spectrometer. Thus, each of the 4 spectra obtained at the end of the acquisition corresponds to an average of 32 scans (average automatically carried out by the equipment).

The 4 spectra obtained over the 4 different positions are then averaged to only give a single average spectrum representative of the scanned industrial tank.

### Acquisition of the Spectra on the “Trial Tank” Samplings

A spectrum in diffuse reflection mode was acquired using an NIRS (for near-infrared spectroscopy) spectrometer in the conditions presented in Table 3, above.

For each tank, 8 different positions are analysed via the NIRS Analyzer Pro spectrometer. Each trial tank is manually moved over these 8 different positions. Thus, each of the 8 spectra obtained at the end of the acquisition corresponds to an average of 32 scans.

The 8 spectra obtained over the 8 different positions are then averaged to only give a single average spectrum representative of the scanned trial tank.

### Reference Analyses

### Reference Analyses on the “Industrial Tank” Samplings

Given that the rearing tanks were prepared manually by introducing components (droppings, live insects, dead insects, nutritive substrate) into the tanks before the scan by the NIRS spectrometer, the quantities of each component are known. Indeed, before the acquisition of the spectra, the mass of each component is measured and expressed as a percentage relative to the total weight of the components introduced into the tank. These values constitute the reference values of each industrial tank.

### Reference Analyses on the “Trial Tank” Samplings

After the acquisition of the spectra, a part (representing ⅛^{th} of each rearing tank) is sorted manually with screens of a different mesh size according to the various components (droppings, live insects, dead insects and contaminants). Once each component has been isolated, the mass of the latter is measured and is expressed as a mass percentage relative to the total weight of the components introduced into the tank, except for the “IMM” KPI which is expressed in milligrams.

### II-Determination of the Quantitative Models

The software Vision used for the acquisition of the spectra mentioned in point I is also used to process the data and develop quantitative models.

### Mathematical Preprocessing of the Spectra

Various mathematical preprocessings were determined to be applied to the spectra obtained in I, such as: first derivative, second derivative, normalisation of the SNV (Standard Normal Variate) type or processing of the Savinsky type.

### Development of the Quantitative Models: Learning Set

After their mathematical preprocessing, the spectra are correlated to the values determined during the reference analyses in point I, via a partial least squares regression or PLS. PLS involves the choice of a certain number of factors (VL): if there are not enough factors, this leads to an under-modelling and to a degradation of the accuracy, and inversely, too many factors lead to an over-modelling and to a degradation of the robustness of the model.

For each performance indicator to be determined (droppings, given population of insects, IMM, nutritive substrate and contaminants) the database (spectra and values determined during the associated reference analyses) was divided into two sets:
a learning set consisting of the spectra and values determined during the associated reference analyses, for 75% of the samples, and
an external validation set consisting of the spectra and values determined during the associated reference analyses, for the rest of the samples (25% of the samples).

Models are established with all of the data of the learning set, by setting the maximum number of factors of the PLS to 16 in the software that then establishes said models by varying the number of factors from 1 to 16. Then, the optimal number of factors to be used for the PLS is determined via a cross-validation. The latter is carried out with the entire learning set.

During the cross-validation, the data for each sample (spectrum and values determined during the associated reference analyses) is removed in turns from the learning set and a model is established with the spectra and values, determined during the associated reference analyses, of the remaining samples:
First of all, the data of a sample, that is to say its spectrum and its associated values determined during the reference analyses, is removed from the learning set then a model is established with the spectra and the associated values determined during the reference analyses of the remaining samples.
Then, the resulting model is used to analyse the spectrum of the sample that was removed, that is to say to predict quantitative values.
Finally, the values predicted by the model (for the spectrum of the removed sample) are subtracted from the values determined during the reference analyses associated with this spectrum, then their differences are squared and summed; this allows to calculate the predicted residual error sum of squares (PRESS) for each factor of the PLS. The PRESS value thus represents the prediction error.
The data for the sample that was removed from the learning set is then reintroduced therein then the above steps are repeated by removing the data for another sample.

The number of factors having a minimal PRESS value is thus determined. This therefore allows to obtain an optimal number of factors to be used for the PLS, for each performance indicator to be determined.

The established models that present the best performance (parameters described in detail below) are kept and their robustness is evaluated on the external validation set.

The various parameters that are used to choose the best models during the creation with the learning set are the following:
R² multiple correlation coefficient: it corresponds to the matching between the data predicted by the model and the values determined during the reference analyses. It must tend towards 1.
SEC standard error of calibration: this value is a statistical parameter, equivalent to a standard deviation and reflecting the upper limit of exactness for the future predictions. It must be as low as possible.
SECV standard error of cross-validation: this value corresponds to the SEC during the cross-validation carried out in the learning set, during the PLS. Preferably, the SECV must have the value as close as possible to the SEC. In order for the model to have an acceptable uncertainty, it was determined that the SECV must be less than or equal to twice the standard deviation associated with the values determined during the reference analyses.

### Development of the Quantitative Models: External Validation

The data of the samples of the external validation set is representative of the range of the values determined during the reference analyses. This data allows to evaluate the robustness of the models developed with the learning set that present the best performance and by studying the following parameters:
R² multiple correlation coefficient: it corresponds to the matching between the data predicted by the model for the external validation set and the values determined during the reference analyses. It must tend towards 1.
SEP standard prediction error: this value is a statistical parameter reflecting the residues (or prediction errors) obtained by the prediction of the external validation set. The ideal case is SEP≤SECV. If this is not the case (SEP>SECV), it must be ensured that the SEP be statistically less than or equal to the SECV (or SECV<SEP≤1.3×SECV).
Bias: the bias is the average value of residues (or of the prediction errors) calculated on the basis of the reference values of the components of the validation set. The bias must be close to 0, which indicates that the deviations are distributed randomly. However, a significant bias value indicates a systematic error, for example such as changes in the equipment, or in the reference analyses. This bias can then be corrected by the software.
RPD: The RPD means “Relative Percent Difference” and is a statistical indicator comparing the precision of the calibration to the variability of the reference data used. This value thus allows to evaluate the quality of the prediction done by the calibration. It is considered that an RPD greater than 1.4 preferably greater than 2 is associated with a good prediction and thus that the model is considered to be sufficient. Inversely, an RPD value lower than 1.4 indicates that the model is not suitable.

The model having the best performance is selected and thus determines the modelling parameters of the final calibration.

### Example 2: Results of the Calibration Trials-Live Insects

### Development of the Model for the Quantification of the Live Insects

The developments of models to determine the quantity of live insects at a specific stage of evolution in a given complex medium (various samplings) were established according to the method described in Example 1.

Thus, a suitable mathematical preprocessing was applied to each spectrum obtained.

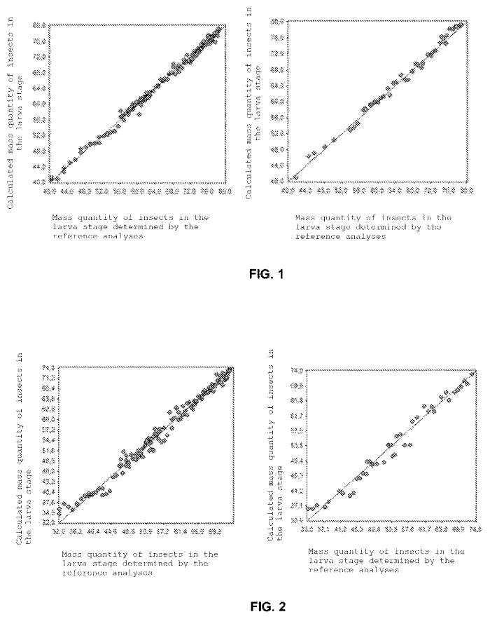

It was determined that the mathematical preprocessing suitable for the performance indicator relative to the insects in the larval stage in the sampling A is a normalisation of the SNV type followed by a first derivative over the spectral range of 1100 to 1600 nm. The statistical parameters of the learning set and of the external validation set allow to approve a model with 13 factors (FIGS. 1, A and B respectively).

It was determined that the mathematical preprocessing suitable for the performance indicator relative to the insects in the larval stage, in the sampling B, is a first derivative over the spectral range of 1100 to 1600 nm. The statistical parameters of the learning set and of the external validation set allow to approve a model with 12 factors (FIGS. 2, A and B respectively).

Moreover, for this same sampling B, it was determined that the mathematical preprocessing suitable for the performance indicator relative to the insects in the nymph stage is a normalisation of the SNV type, followed by a second derivative over the spectral range of 1100 to 1600 nm. The statistical parameters of the learning set and of the external validation set allow to approve a model with 9 factors (FIGS. 3, A and B respectively).

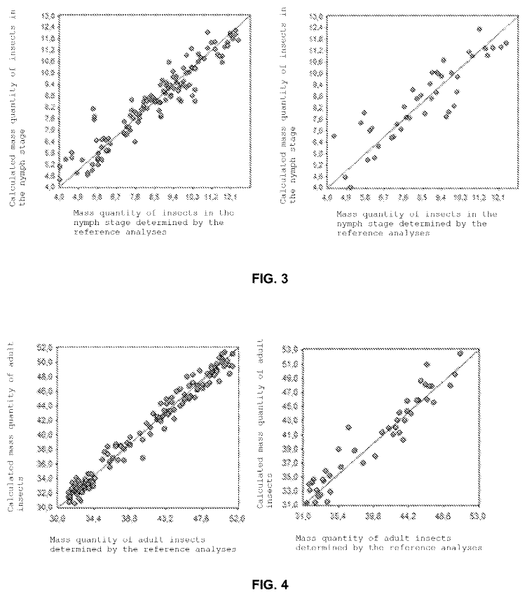

It was determined that the mathematical preprocessing suitable for the performance indicator relative to the insects in the adult stage in the sampling C is a first derivative over the spectral range of 1100 to 1600 nm. The statistical parameters of the learning set and of the external validation set allow to approve a model with 11 factors (FIGS. 4, A and B respectively).

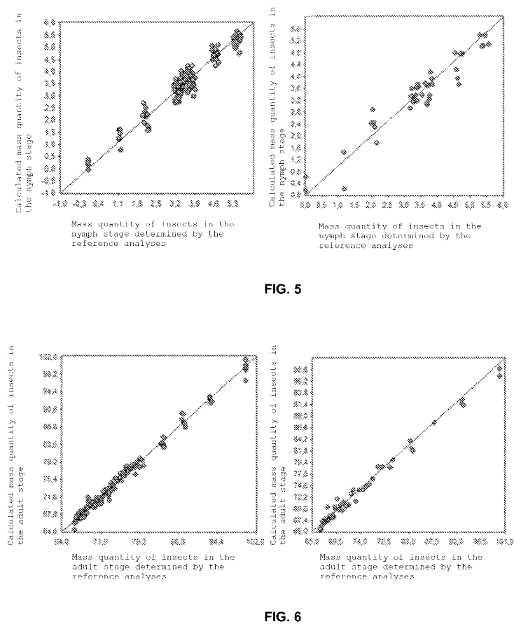

In this same sampling C, it was determined that the mathematical preprocessing suitable for the performance indicator relative to the insects in the nymph stage is a normalisation of the SNV type, followed by a second derivative over the spectral range of 1100 nm to 1600 nm. The statistical parameters of the learning set and of the external validation set allow to approve a model with 8 factors (FIGS. 5, A and B respectively).

Finally, it was determined that the mathematical preprocessing suitable for the performance indicator relative to the insects in the adult stage in the sampling D is a first derivative over the spectral range of 1100 to 1600 nm. The statistical parameters of the learning set and of the external validation set allow to approve a model with 11 factors (FIGS. 6, A and B respectively).

A summary of the results is presented in Table 4 below.

TABLE 4

| | | Calibration model | | | Validation model | | | | |
|---|---|---|---|---|---|---|---|---|---|
| Sampling | KPI | R² | SEC | SECV | R² | SEP | Bias | Uncertainty | RPD |
| A | Larvae | 0.9936 | 0.8472 | 1.3096 | 0.995 | 1.0022 | −0.4036 | 2.62 | 7.62 |
| B | Larvae | 0.9828 | 1.5456 | 1.874 | 0.987 | 1.8005 | 0.2876 | 3.75 | 6.02 |
| | Nymphs | 0.9 | 0.6936 | 0.939 | 0.8883 | 0.969 | −0.0757 | 1.88 | 2.25 |
| C | Adults | 0.9745 | 1.1028 | 1.5454 | 0.9568 | 1.8337 | −0.4874 | 3.09 | 4.18 |
| | Nymphs | 0.9398 | 0.3237 | 0.3994 | 0.9556 | 0.3874 | 0.0456 | 0.80 | 3.22 |
| D | Adults | 0.9909 | 0.9336 | 1.3278 | 0.9946 | 1.0483 | 0.3359 | 2.66 | 7.02 |

It is noted that in the case B above, if the larvae constitute the population of live insects in the sense of the present invention then the nymphs are a component of the complex medium as insects at at least one different stage of development than at least one insect of the population of live insects, or vice versa. Likewise, in the case C above, if the nymphs constitute the population of live insects in the sense of the present invention then the adults are a component of the complex medium as insects at at least one different stage of development than at least one insect of the population of live insects, or vice versa. These two populations consist, however, of live insects.

In conclusion, for each model obtained, the R-correlation coefficients of the learning set on the one hand and of the external validation set on the other hand are very high.

The correlation between the values calculated and the values determined by the reference analyses, according to each model developed, is very high.

For each model relative to a given population of live insects, the RPD values are always greater than 2 and are very high thus pointing to the good quality of prediction of the models over the ranges developed.

The measurement uncertainty represents the maximum error associated with the prediction of the mass quantity of a given population of live insects. The values obtained are relatively low.

These results confirm that the analysis by NIRS spectrometry is a suitable method for determining a quantitative value of a population of live insects in a complex medium comprising in particular droppings, substrate and/or dead insects.

Moreover, the results indicate that the method according to the method of the invention allows to distinguish various populations of live insects even though the latter are mobile in the complex medium, without previous manipulation of the sample.

The results of the sampling D demonstrate that the method according to the method of the invention can be used when the insects are reared on metal gratings and thus that the presence of a metal grating does not affect the results.

### Example 3: Results of the Calibration Trials—Droppings

### Development of the Model for the Quantification of the Droppings

The development of models to determine the quantity of droppings was carried out according to the method described in Example 1.

A suitable mathematical preprocessing is applied to each spectrum obtained.

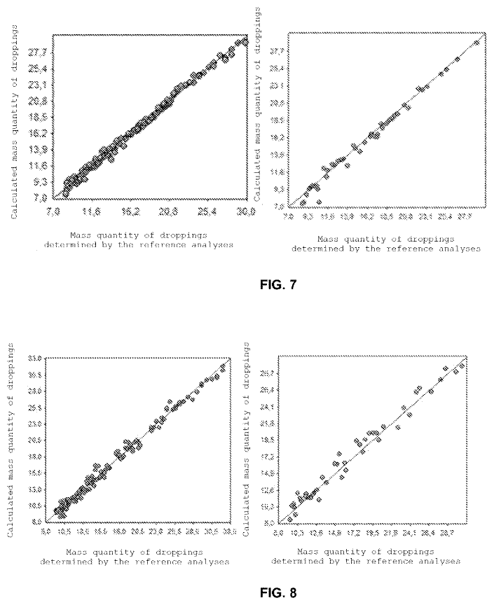

In particular, it was determined that the mathematical preprocessing suitable for the performance indicator relative to the droppings in the sampling A is a normalisation of the SNV type followed by a second derivative over the spectral range of 1100 to 1600 nm. The statistical parameters of the learning set and of the external validation set allow to approve a model with 9 factors (FIGS. 7, A and B respectively).

It was determined that the mathematical preprocessing suitable for the performance indicator relative to the droppings in the sampling B is a normalisation of the SNV type followed by first derivative over the spectral range of 1100 nm to 1600 nm. The statistical parameters of the learning set and of the external validation set allow to approve a model with 14 factors (FIGS. 8, A and B respectively).

A summary of the results is presented in Table 5 below.

TABLE 5

| | | Calibration model | | | Validation model | | | | |
|---|---|---|---|---|---|---|---|---|---|
| Sampling | KPI | R² | SEC | SECV | R² | SEP | Bias | Uncertainty | RPD |
| A | Droppings | 0.9945 | 0.4394 | 0.5799 | 0.9931 | 0.6918 | 0.2878 | 1.16 | 9.65 |
| B | Droppings | 0.9897 | 0.6714 | 1.1017 | 0.9877 | 1.0033 | −0.2975 | 2.20 | 5.63 |

In conclusion, for each model obtained, the R² correlation coefficients of the learning set on the one hand and of the external validation set on the other hand are very high.

The correlation between the values calculated and the values determined by the reference analyses, according to each model developed, is very high.

The RPD values are always greater than 2 and are very high thus pointing to the good quality of prediction of the models over the ranges developed.

The measurement uncertainty represents the maximum error associated with the prediction of the mass quantity of droppings. The values obtained are relatively low.

These results confirm that the analysis by NIRS spectrometry is a suitable method for determining a quantitative value of insect droppings in a complex medium comprising in particular live insects at various stages of evolution, nutritive substrate and/or dead insects.

### Example 4: Results of the Calibration Trials-Nutritive Substrate

### Development of the Model for the Quantification of a Nutritive Substrate

The development of models to determine the quantity of nutritive substrate in a complex medium was carried out according to the method described in Example 1.

A suitable mathematical preprocessing is applied to each spectrum obtained.

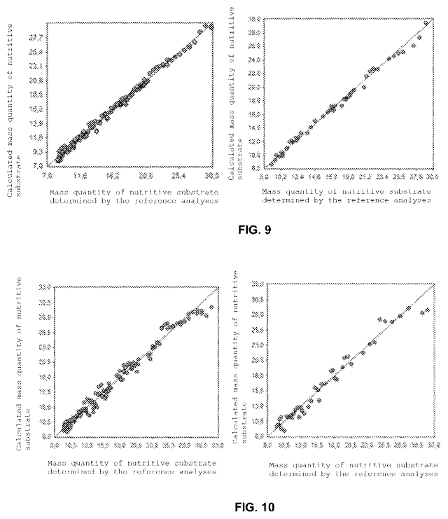

It was determined that the mathematical preprocessing suitable for the performance indicator relative to the nutritive substrate in the sampling A is a normalisation of the SNV type, followed by a second derivative over the spectral range of 1100 to 1600 nm. The statistical parameters of the learning set and of the external validation set allow to approve a model with 10 factors (FIGS. 9, A and B respectively).

It was determined that the mathematical preprocessing suitable for the performance indicator relative to the nutritive substrate in the sampling B is a normalisation of the SNV type, followed by first derivative over the spectral range of 1100 to 1600 nm. The statistical parameters of the learning set and of the external validation set allow to approve a model with 10 factors (FIGS. 10, A and B respectively).

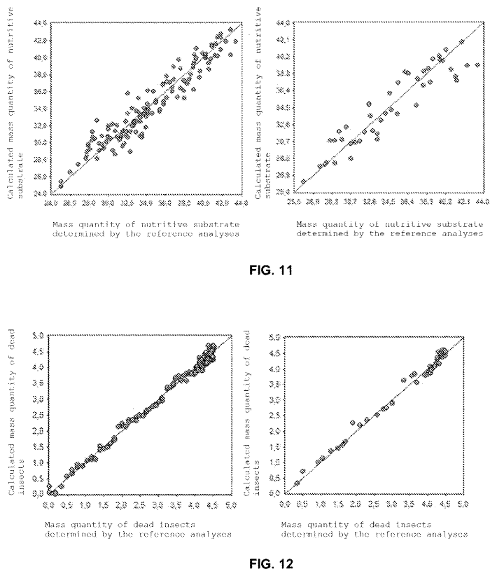

With regard to the sampling C, after research, it was determined that the suitable mathematical preprocessing is a first derivative over the spectral range of 1100 to 1600 nm. The statistical parameters of the learning set and of the external validation set allow to approve a model with 10 factors (FIGS. 11, A and B respectively)

A summary of the results is presented in Table 6 below.

TABLE 6

| | | Calibration model | | | Validation model | | | | |
|---|---|---|---|---|---|---|---|---|---|
| Sampling | KPI | R² | SEC | SECV | R² | SEP | Bias | Uncertainty | RPD |
| A | Nutritive | 0.9941 | 0.4431 | 0.6565 | 0.997 | 0.4668 | 0.0645 | 1.31 | 8.53 |
| | substrate | | | | | | | | |
| B | Nutritive | 0.9815 | 0.8742 | 1.148 | 0.9846 | 1.1156 | 0.1301 | 2.3 | 5.41 |
| | substrate | | | | | | | | |
| C | Nutritive | 0.9078 | 1.4189 | 1.9498 | 0.9304 | 1.6883 | 0.1056 | 3.90 | 2.32 |
| | substrate | | | | | | | | |

For each model obtained, the R² correlation coefficients of the learning set on the one hand and of the external validation set on the other hand are very high.

For each model relative to the nutritive substrate, the RPD values are always greater than 2 and are very high thus pointing to the good quality of prediction of the models over the ranges developed.

The measurement uncertainty represents the maximum error associated with the prediction of the mass quantity of nutritive substrate. The values obtained are relatively low.

These results confirm that the analysis by NIRS spectrometry is a suitable method for determining in a reliable manner a quantitative value of a nutritive substrate in various complex mediums comprising live insects at various stages of evolution, droppings and/or dead insects.

### Example 5: Results of the Calibration Trials-Dead Insects

### Development of the Model for the Quantification of Dead Insects

The development of models to determine the quantity of dead insects in a complex medium was carried out according to the method described in Example 1.

A suitable mathematical preprocessing is applied to each spectrum obtained.

It was determined that the mathematical preprocessing suitable for the spectra coming from the sampling A is a normalisation of the SNV type, followed by a first derivative over the spectral range of 1100 to 1600 nm. The statistical parameters of the learning set and of the external validation set allow to approve a model with 11 factors (FIGS. 12, A and B respectively).

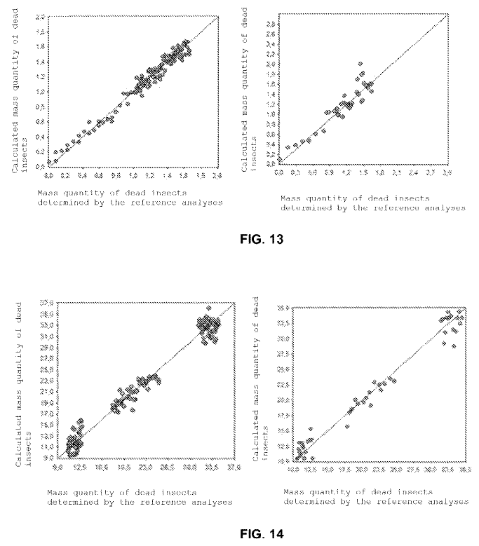

It was determined that the mathematical preprocessing suitable for the spectra coming from the sampling B is a normalisation of the SNV type, followed by a second derivative over the spectral range of 1100 to 1600 nm. The statistical parameters of the learning set and of the external validation set allow to approve a model with 13 factors (FIGS. 13, A and B respectively).

It was determined that the suitable mathematical preprocessing of the spectra acquired on the sampling C is a second derivative over the spectral range of 1100 to 1600 nm. The statistical parameters of the learning set and of the external validation set allow to approve a model with 6 factors (FIGS. 14, A and B respectively).

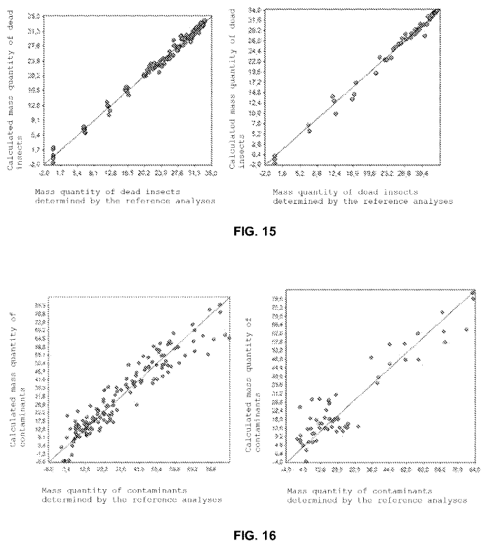

Finally, it was determined that the suitable mathematical preprocessing of the spectra acquired on the sampling D is a normalisation of the SNV type, followed by a first derivative over the spectral range of 1100 to 1600 nm. The statistical parameters of the learning set and of the external validation set allow to approve a model with 10 factors (FIGS. 15, A and B respectively).

A summary of the results is presented in Table 7 below.

TABLE 7

| | | Calibration model | | | Validation model | | | | |
|---|---|---|---|---|---|---|---|---|---|
| Sampling | KPI | R² | SEC | SECV | R² | SEP | Bias | Uncertainty | RPD |
| A | Dead | 0.9914 | 0.1254 | 0.1884 | 0.9904 | 0.1835 | −0.0513 | 0.38 | 6.81 |
| | insects | | | | | | | | |
| B | Dead | 0.9681 | 0.072 | 0.1394 | 0.9273 | 0.1606 | −0.036 | 0.28 | 2.77 |
| | insects | | | | | | | | |
| C | Dead | 0.973 | 1.4862 | 1.6046 | 0.9862 | 1.4814 | 0.0696 | 3.21 | 5.51 |
| | insects | | | | | | | | |
| D | Dead | 0.9924 | 0.8393 | 1.3092 | 0.9946 | 1.0755 | 0.0963 | 2.62 | 7.12 |
| | insects | | | | | | | | |

In conclusion, for each model obtained, the R² correlation coefficients of the learning set on the one hand and of the external validation set on the other hand are very high.

For each model relative to the dead insects, the RPD values are always greater than 2 and are very high thus pointing to the good quality of prediction of the models over the ranges developed.

The measurement uncertainty represents the maximum error associated with the prediction of the quantity of dead insects. The values obtained are relatively low.

These results confirm that the analysis by NIRS spectrometry is a suitable method for determining in a reliable manner the quantitative value of dead insects in a complex medium comprising in particular live insects at various stages of evolution, nutritive substrate and/or droppings.

Moreover, these results demonstrate that the analysis by NIRS spectrometry is a suitable method for distinguishing dead insects from the live insects in a complex medium.

### Example 6: Results of the Calibration Trials-Contaminants

### Development of the Model for the Quantification of the Contaminants

The development of models to determine the quantity of contaminants in a complex medium was carried out according to the method described in Example 1.

It was determined that the suitable mathematical preprocessing of the spectra acquired from the sampling E is a processing of the Savinsky type, followed by a first derivative over the spectral range of 1100 to 1600 nm. The statistical parameters of the learning set and of the external validation set allow to approve a model with 12 factors (FIGS. 16, A and B respectively).

A summary of the results is presented in Table 8 below.

TABLE 8

| | | Calibration model | | | Validation model | | | | |
|---|---|---|---|---|---|---|---|---|---|
| Sampling | KPI | R² | SEC | SECV | R² | SEP | Bias | Uncertainty | RPD |
| E | Contaminants | 0.9023 | 7.170 | 8.775 | 0.929 | 8.302 | −1.39 | 15.68 | 2.85 |

In conclusion, the R² correlation coefficients of the learning set on the one hand and of the external validation set on the other hand are very high.

The correlation between the calculated values and the values determined by the reference analyses is very strong.

In the model relative to the contaminants, the RPD value is greater than 2 which points to the good quality of prediction of the model over the ranges developed.

These results confirm that the analysis by NIRS spectrometry is a suitable method for determining in a reliable manner the quantitative value of contaminants in a complex medium comprising in particular live insects, nutritive substrate and droppings.

### Example 7: Results of the Calibration Trials—IMM

Development of the Model for the Determination of the Individual Mean Mass in the Range from 1.03 mg to 42.86 mg

The development of models to determine the individual mean mass of the insects in a complex medium was carried out according to the method described in Example 1.

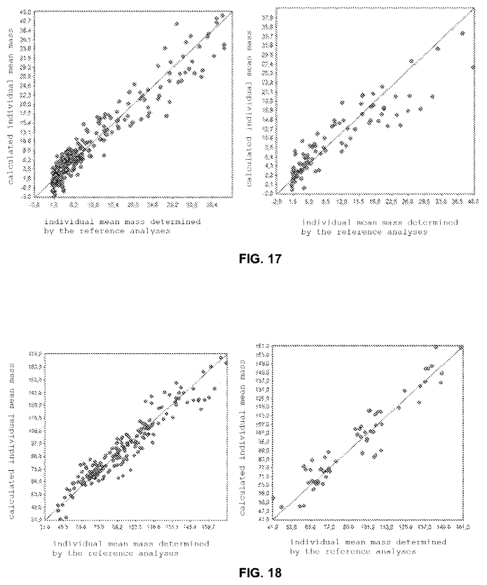

In particular, it was determined that the mathematical preprocessing suitable for the IMM indicator in the sampling E in the range from 1.03 mg to 42.86 mg is a first derivative over the spectral range going from 1100 to 1600 nm. The statistical parameters of the learning set and of the external validation set allow to approve a model with 14 factors (FIGS. 17, A and B respectively).

A summary of the results is presented in Table 9 below.

TABLE 9

| | | Calibration model | | | Validation model | | | | |
|---|---|---|---|---|---|---|---|---|---|
| Sampling | KPI | R² | SEC | SECV | R² | SEP | Bias | Uncertainty | RPD |
| E | IMM | 0.922 | 2.8295 | 3.276 | 0.9133 | 3.8428 | 0.5073 | 6.56 | 2.87 |
| | (1.03 | | | | | | | | |
| | mg to | | | | | | | | |
| | 42.86 | | | | | | | | |
| | mg) | | | | | | | | |

In conclusion, the R² correlation coefficients of the learning set on the one hand and of the external validation set on the other hand are very high.

Indeed, the correlation between the calculated values and the values determined by the reference analyses is very strong.

In the model relative to the determination of the individual mean mass of the insects, the RPD value is greater than 2 which points to the good quality of prediction of the model over the range from 1.03 mg to 42.86 mg.

Development of the Model for the Determination of the Individual Mean Mass in the Range from 40.85 mg to 173.46 mg

The development of models to determine the individual mean mass of the insects in a complex medium was carried out according to the method described in Example 1.

It was determined that the mathematical preprocessing suitable for the IMM indicator in the sampling E for the development of a model in the range from 40.85 mg to 173.46 mg is a normalisation of the SNV type followed by a first derivative over the spectral range going from 1100 to 1600 nm. The statistical parameters of the learning set and of the external validation set allow to approve a model with 15 factors (FIGS. 18, A and B respectively).

A summary of the results is presented in Table 10 below.

TABLE 10

| | | Calibration model | | | Validation model | | | | |
|---|---|---|---|---|---|---|---|---|---|
| Sampling | KPI | R² | SEC | SECV | R² | SEP | Bias | Uncertainty | RPD |
| E | IMM | 0.905 | 9.281 | 11.68 | 0.952 | 8.902 | −1.547 | 21.54 | 2.66 |
| | (40.85 | | | | | | | | |
| | mg to | | | | | | | | |
| | 173.46 | | | | | | | | |
| | mg) | | | | | | | | |

In conclusion, the R² correlation coefficients of the learning set on the one hand and of the external validation set on the other hand are very high.

Indeed, the correlation between the calculated values and the values determined by the reference analyses is very strong.

In the model relative to the determination of the individual mean mass of the insects, the RPD value is greater than 2 which points to the good quality of prediction of the model over the range from 40.85 mg to 173.46 mg.

These results confirm that analysis the by NIRS spectrometry is a suitable method for determining the individual mean mass of the insects in a complex medium comprising in particular live insects at various stages of evolution, nutritive substrate and/or dead insects, in a non-invasive manner.

1. Non-destructive method for quantitatively determining at least one performance indicator of a rearing of a population of live insects in a complex medium, said method comprising the following steps:
(i) a step of irradiating the complex medium including the population of live insects with a light including one or more wavelengths comprised in a range going from 420 nm to 2500 nm,
(ii) a step of collecting the light reflected by the complex medium in order to obtain a reflectance spectrum of the medium, and
(iii) a step of correlating said spectrum to a quantitative value of at least one performance indicator.

2. Method according to claim 1, wherein the complex medium comprises at least two different components other than the population of live insects.

3. Method according to claim 2, wherein the at least two different components are chosen from a substrate and droppings.

4. Method according to claim 1, wherein the complex medium comprises at least one insect at at least one different stage of development than at least one insect of the population of live insects.

5. Method according to claim 1, wherein the complex medium comprises at least one dead insect.

6. Method according to claim 1, wherein the performance indicator is chosen from a quantification of a population of insects, of a substrate, of the droppings, of a contaminant, and/or an individual mean mass of the insects.

7. Method according to claim 6, wherein the performance indicator is a quantification of the droppings.

8. Method according to claim 6, wherein the performance indicator is a quantification of a population of insects.

9. Method according to claim 8, wherein the population of insects consists of the dead insects.

10. Method according to claim 8, wherein the population of insects consists of live insects at a given stage of development.

11. Method according to claim 1, further including, after the step (ii) of collection and before the step (iii) of correlation, a step of mathematical processing of the reflectance spectrum comprising a first derivative, a second derivative, a processing of the Savinsky type, and/or a normalisation of the SNV type.

12. A method of monitoring of growth of the population of live insects to determine the efficiency of a sorting machine and/or to determine the efficiency of the equipment for feeding the population of live insects comprising employing the method of claim 1.

13. A method for the quantitative determination of at least one performance indicator of a population of live insects in a complex medium comprising employing a spectrophotometer.

14. Method according to claim 13, wherein the performance indicator is chosen from a quantification of a population of insects, of a substrate, of the droppings, of a contaminant, and/or an individual mean mass of the insects.
